(19) United States
(12) Patent Application Publication
(10) Pub. No.: US 2020/0398219 A1
(43) Pub. Date: Dec. 24, 2020

(54) BIOREACTOR FOR CONVERTING GASEOUS CO2

(71) Applicant: INWOO CORPORATION, Seoul (KR)

(72) Inventor: Hyun Yong SHIN, Seoul (KR)

(21) Appl. No.: 16/968,424

(22) PCT Filed: Feb. 1, 2019
(86) PCT No.: PCT/KR2019/001415

§ 371 Date: Aug. 7, 2020

(30) Foreign Application Priority Data
Feb. 9, 2018 (NL) 2020407

(51) Int. Cl. B01D 53/84; B01D 53/62; B01D 53/96; C12P 7/54; C12P 7/46; C12M 1/107; C12M 1/12

(52) U.S. Cl. CPC B01D 53/84; B01D 53/62; B01D 53/96; C12P 7/54; B01D 2258/05; C12M 21/04; C12M 25/06; B01D 2251/95; B01D 2257/504; C12P 7/46

(57) ABSTRACT

The invention relates to a bioreactor for the anaerobic conversion of gaseous CO₂ and a liquid culture medium to organic acids. The invention further relates to a process for the anaerobic conversion of gaseous CO₂ and liquid culture medium to organic acids, using said bioreactor. The invention further relates to a process for the anaerobic conversion of gaseous CO₂ and liquid culture medium to gaseous CH₄ using the organic acids as intermediate products, using said bioreactor and an anaerobic digester.

### TECHNICAL FIELD

The invention relates to a bioreactor for the anaerobic conversion of gaseous CO₂ and a liquid culture medium to organic acids. The invention further relates to a process for the anaerobic conversion of gaseous CO₂ and liquid culture medium to organic acids, using said bioreactor. The invention further relates to a process for the anaerobic conversion of gaseous CO₂ and liquid culture medium to gaseous CH₄ using the organic acids as intermediate products, using said bioreactor and an anaerobic digester.

### BACKGROUND ART

The global energy demand is growing rapidly. The major part of this demand is still met by employing fossil fuels. Due to, amongst others, the use of fossil fuels, the concentration of greenhouse gases in the atmosphere is rising rapidly, with carbon dioxide (CO₂) emissions originating from fossil fuels being the most important contributor. In order to minimize related global warming, the emission of greenhouse gases, particularly of CO₂, must be reduced. One way of addressing this problem is converting the CO₂ originating from fossil fuels into valuable chemicals instead of releasing it to the atmosphere. Another way is the use of bioenergy from biogas or bioethanol obtained from renewable energy sources such as biomass as a fuel source instead of fossil fuels. Biogas produced through anaerobic digestion of biomass is roughly composed of 50-75% of methane (CH₄) and 25-50% of CO₂. In order to be suitable as a vehicle fuel or for grid injection, the CH₄ must be purified and upgraded to enrich the CH₄-content and to improve the energy content of the biogas.

The prior art describes processes for the conversion of CO₂ in biogas and/or in flue gases into organic acids using liquid culture media and anaerobic organic-acid producing microorganisms.

FR3048366A1 discloses a process comprising the steps of producing biogas from organic material, purifying the biogas to a gas comprising CH₄ and CO₂ and contacting the gas comprising CH₄ and CO₂ with enzymes or microorganisms to obtain a biogas depleted in CO₂ and a fuel or an intermediate product for the production of a fuel. The enzymes or microorganisms are comprised in a gel. The microorganisms can be Actinobacillus succinogenes. FR3048366A1 also relates to a plant for purifying a biogas stream comprising CH₄ and CO₂, said plant comprising a methanizing device for converting organic material into biogas, a biogas purification device producing a CO₂-gas stream and a CH₄-gas stream, and a conduit for discharging the CO₂-gas stream from the purification device, said conduit comprising a gel comprising enzymes or microorganisms for converting CO₂-gas stream into a fuel or an intermediate product necessary for the formation of a fuel. FR3048366A1 further discloses a plant for purifying a biogas stream comprising CH₄ and CO₂, comprising a methanizing device for converting organic material into biogas, a conduit for discharging the biogas to a purification device, said conduit comprising a gel comprising enzymes or microorganisms for converting the biogas into a fuel or an intermediate product necessary for the formation of a fuel and a biogas depleted in CO₂, and a device for purifying the biogas depleted in CO₂.

WO2014/188000A1 concerns a method for upgrading fuel gas and for the production of succinic acid comprising the steps of:

a) providing a bioreactor, anaerobic succinic acid-producing microorganisms, and a carbon based substrate for said anaerobic succinic acid producing microorganisms,

b) adding a CO₂-containing gas to the bioreactor,

c) collecting the upgraded gas thus produced, wherein said upgraded gas has a lower CO₂ content than the CO₂-containing gas added, and

d) collecting the effluent containing succinic acid

It is described in WO2014/188000A1 that the bioreactor can be a continuous stirred-tank reactor (CSTR) for comprising a liquid fermentation broth and a gas injection system for injecting CO₂-containing gas into the liquid fermentation broth.

I. B. Gunnarsson et al, Environmental Science and Technology, 2014, 48, pp 12464-12468, disclose that Actinobacillus succinogenes can produce succinic acid using CO₂ from biogas and a carbon source. Gunnarsson et al. describe a stirred bioreactor comprising a liquid fermentation broth containing Actinobacillus succinogenes and a gas injection system for injection of CO₂-containing gas at the bottom of the reactor into the liquid phase. Gas was recirculated over the liquid phase of the stirred bioreactor during fermentation. It is described that the conversion capacity of the bioreactor depends on the solubility of CO₂ in the liquid phase at the CO₂ partial pressure.

### DISCLOSURE OF INVENTION

### Technical Problem

There is a need for an improved process for the anaerobic conversion of gaseous CO₂, such as the CO₂ in biogas, and a liquid culture medium into organic acids. Moreover, there is a need for a bioreactor for use in such an improved process. In particular, there is a need for a process for the anaerobic conversion of gaseous CO₂ and a liquid culture medium into organic acids, and a bioreactor for use in said process, wherein the process has improved capture of CO₂ by anaerobic organic-acid producing microorganisms, an improved utilization of CO₂ by anaerobic organic-acid producing microorganisms and/or an improved scalability. In addition, there is a need for an improved process for the anaerobic conversion of gaseous CO₂ and a liquid culture medium to gaseous CH₄ using the organic acids as intermediate products.

### Solution to Problem

The inventor has found that one or more of the above objects can be met by introducing CO₂-containing gas in a bioreactor and by introducing a liquid culture medium at the top of a bioreactor, wherein said bioreactor contains at least one perforated plate comprising on its upper surface anaerobic organic acid-producing microorganisms. The perforations in the one or more plates allow the liquid culture medium to flow downwards in the bioreactor over the anaerobic organic acid-producing microorganisms on the at least one perforated plates. The CO₂-containing gas can freely move through the perforations and can freely contact the organic acid-producing microorganisms, hardly limited by the solubility of CO₂ in the liquid culture medium. The process is scalable by using more perforated plates with anaerobic organic acid-producing microorganisms, by using more than one bioreactor and/or by using a larger bioreactor.

Accordingly, in a first aspect, the invention relates to a bioreactor (1) for the anaerobic conversion of gaseous CO₂ and a liquid culture medium to organic acids, said bioreactor (1) comprising a cavity (2a), an outer wall (2b), a bottom (2c) and a top (2d),

wherein the cavity (2a) comprises at least one plate (3) having at least one perforation (4),

wherein said at least one plate (3) is positioned perpendicularly to the outer wall (2b),

wherein said bioreactor (1) further comprising a pipe (5) connected to a first liquid outlet (6) located at the bottom (2c) of the bioreactor (1) for discharging liquid, a pipe (7) connected to a second liquid outlet (8) located at the bottom (2c) of the bioreactor (1) and to the inlet of a first pump (9), a pipe (10) connected to an outlet of the first pump (9) and to a first liquid inlet (11) located at the top (2d) of the bioreactor (1) for recycling liquid culture medium over the at least one plate (3), a pipe (13) connected to a first gas inlet (12) for providing CO₂-containing gas to the bioreactor (1), a pipe (15) connected to a first gas outlet (14) for discharging gas from the bioreactor (1), and a pipe (17) connected to a second liquid inlet (16) for supplying fresh liquid culture medium to the bioreactor (1).

Preferably, the at least one plate (3) comprises on its upper surface anaerobic organic acid-producing microorganisms.

In a second aspect, the invention relates to a method for the anaerobic conversion of gaseous CO₂ and liquid culture medium to organic acids, said method comprising the steps of:

(a) providing a bioreactor (1) as defined hereinbefore;

(b) adding anaerobic organic acid-producing microorganisms to the upper surface of the plate (3) or to at least the upper surface of the most upper plate (3) of the bioreactor (1);

(c) adding fresh liquid culture medium via pipe (17) and second liquid inlet (16) and

CO₂-containing gas via pipe (13) and first gas inlet (12) to the bioreactor (1);

(d) circulating liquid culture medium over the one or more plates (3) by collecting the liquid carbohydrate medium at second liquid outlet (8) located at the bottom (2c) of the bioreactor (1) and supplying it to the first liquid inlet (11) located at the top (2d) of the bioreactor via pipe (7), first pump (9) and pipe (10), to obtain an organic acid-containing liquid medium and a gas depleted in CO₂;

(e) discharging the organic acid-containing liquid medium obtained in step (d) via the first liquid outlet (6) and pipe (5); and

(f) discharging the gas depleted in CO₂ obtained in step (d) via the first gas outlet (14) and pipe (15).

### Advantageous Effects of Invention

The inventor has found that the organic acid-containing liquid medium produced in the bioreactor can be supplied to an anaerobic digester where it is subsequently converted to CH₄. Hence, if the CO₂-containing gas supplied to the bioreactor is CH₄-containing biogas originating from an anaerobic digester, wherein organic material is digested, and the organic acid-containing liquid medium produced in the bioreactor is subsequently supplied to that digester where it is converted to CH₄, a larger fraction of the organic material originally present in the digester is converted to CH₄.

### BRIEF DESCRIPTION OF DRAWINGS

FIG. 1 schematically depicts a bioreactor according to the invention for the anaerobic conversion of gaseous CO₂ and a liquid culture medium to organic acids.

FIG. 2 schematically depicts a continuous stirred-tank reactor (CSTR) for the anaerobic conversion of gaseous CO₂ and a liquid culture medium to organic acids.

FIG. 3 shows the consumption of CO₂ as a function of time in the bioreactor of FIG. 1 and in the CSTR of FIG. 2.

FIG. 4 shows the acid concentration obtained in the bioreactor of FIG. 1 with three different liquid culture media.

FIG. 5 schematically depicts a biogas production facility comprising a digester for the production of biogas from organic material and an interconnected bioreactor according to the invention.

FIG. 6 shows the daily CH₄ production of an anaerobic digester and of the biogas production facility of FIG. 5.

### BEST MODE FOR CARRYING OUT THE INVENTION

In a first aspect the invention provides a bioreactor (1) for the anaerobic conversion of gaseous CO₂ and a liquid culture medium to organic acids, said bioreactor (1) comprising a cavity (2a), an outer wall (2b), a bottom (2c) and a top (2d),

wherein the cavity (2a) comprises at least one plate (3) having at least one perforation (4), wherein said at least one plate (3) is positioned perpendicularly to the outer wall (2b),

wherein said bioreactor (1) further comprising a pipe (5) connected to a first liquid outlet (6) located at the bottom (2c) of the bioreactor (1) for discharging liquid, a pipe (7) connected to a second liquid outlet (8) located at the bottom (2c) of the bioreactor (1) and to the inlet of a first pump (9), a pipe (10) connected to an outlet of the first pump (9) and to a first liquid inlet (11) located at the top (2d) of the bioreactor (1) for recycling liquid culture medium over the at least one plate (3), a pipe (13) connected to a first gas inlet (12) for providing CO₂-containing gas to the bioreactor (1), a pipe (15) connected to a first gas outlet (14) for discharging gas from the bioreactor (1), and a pipe (17) connected to a second liquid inlet (16) for supplying fresh liquid culture medium to the bioreactor (1).

In a preferred embodiment, the at least one plate (3) of the bioreactor (1) comprises on its upper surface anaerobic organic acid-producing microorganisms.

The bioreactor (1) is used for the anaerobic conversion of gaseous CO₂ and a liquid culture medium to organic acids. Hence, the bioreactor (1) is suitable for operation under conditions free from atmospheric oxygen In order words, the bioreactor (1) can be operated leak tight or leak proof.

The bioreactor (1) as defined hereinbefore is not particular limited as regards the number of plates (3). The higher the outer wall (2b), the higher the number of plates (3) can be. The more plates (3) are used, the higher the amount of microorganisms that can be comprised by the bioreactor (1) and the higher the capacity of the bioreactor (1) to convert gaseous CO₂ and a liquid culture medium to organic acids. In a particular embodiment, the number of plates (3) having at least one perforation (4) ranges from 2 to 500. The plates (3) are advantageously spaced between 0.5 and 5 cm apart from each other, such as for example 2 cm.

The bioreactor (1) as defined hereinbefore is not particular limited as regards its size. In an embodiment, the outer wall (2b) is between 0.5 m and 10 meter high and the number of plates (3) in the bioreactor (1) ranges between 2 and 500.

The plates (3) can be advantageously be made of metal, such as stainless steel, glass or plastic.

Every plate (3) of the bioreactor (1) can comprise on its upper surface anaerobic organic acid-producing microorganisms These microorganisms can for example be applied to the at least one plate (3) by spraying a liquid suspension with microorganisms onto the at least one plate (3). During operation of the bioreactor (1), the population of the anaerobic organic acid producing microorganisms on the one or more plates (3) grows, thereby establishing a biofilm. If the bioreactor (1) comprises more than one plate (3), spraying a liquid suspension with microorganism onto the most upper plate (3) is sufficient to establish a biofilm on every plate (3) since recirculation of liquid over the bioreactor causes microorganisms to contact every plate (3).

As described hereinabove, the bioreactor (1) comprises a first liquid inlet (11) located at the top (2d) of the bioreactor (1) and a first gas inlet (12) for providing CO₂-containing gas to the bioreactor (1). Every plate (3) has at least one perforation (4). This at least one perforation allows the liquid entering the bioreactor at the top side (2d) to move to the bottom side (2c). Moreover, the at least one perforation (4) allows CO₂-containing gas to freely distribute across the bioreactor (1).

If the bioreactor (1) comprises more than one plate (3), the at least one perforations (4) of different plates (3) are preferably not arranged in one vertical line. In other words, if the bioreactor (1) comprises more than one plate (3), the at least one perforations (4) of different plates (3) are preferably not arranged exactly below one another. The reason is as follows. As will be understood by one skilled in the art, when the bioreactor (1) is in operation, the anaerobic organic acid-producing microorganisms convert gaseous CO₂ to organic acids using the liquid culture medium This means that the liquid culture medium, entering the bioreactor (1) at the top side (2d), should be able to reach the microorganisms on every plate (3). If the at least one perforations (4) of different plates (3) are arranged exactly below one another, the liquid culture medium only reaches the microorganisms on the most upper plate (3) and subsequently drips down to the bottom (2c) of the bioreactor (1) without reaching microorganisms on other plates (3).

In a preferred embodiment, the at least one plate (3) contains multiple perforations (4), such as more than 10, 100, 500 or 1000. In another preferred embodiment, the at least one plate (3) has multiple perforations (4) and is a grid or a mesh screen.

The perforation or perforations (4) preferably have a size of between 0.5 and 100 mm, more preferably between 1 and 2 mm. The perforations (4) are not particularly limited as regards their form The perforations (4) can for example be square, triangular, circular or oval.

Another preferred embodiment concerns a biogas production facility comprising a digester (20) for the anaerobic production of CO₂-containing biogas from organic material and at least one bioreactor (1) as defined hereinbefore, said digester (20) comprising a gas outlet (21) connected to pipe (13) of the at least one bioreactor (1) for supplying CO₂-containing

biogas to the at least one bioreactor (1) and a liquid inlet (22) connected to pipe (5) of the at least one bioreactor (1) for supplying organic acid-containing liquid medium to the digester (20) via a second pump (23). Digesters for the anaerobic conversion of organic material into CH₄- and CO₂-containing biogas are well-known in the art. In this respect, reference is made to WO2011/138426A1.

In a preferred embodiment, the at least one plate (3) in every bioreactor (1) of the biogas production facility as defined hereinbefore comprises on its upper surface anaerobic organic acid-producing microorganisms.

Dependent on the capacity of the digester (20) and the size of the bioreactor (1), the biogas production facility can comprise more than one bioreactor (1), such as 2 to bioreactors (1). If the biogas production facility comprises more than one bioreactor (1), the bioreactors are

preferably connected to the digester (20) in parallel via separate pipes (5) and (13), and pumps (23).

In a second aspect, the invention provides a method for the anaerobic conversion of gaseous CO₂ and liquid culture medium to organic acids, said method comprising the steps of:

(a) providing a bioreactor (1) as defined hereinbefore;

(b) adding anaerobic organic acid-producing microorganisms to the upper surface of the plate (3) or to at least the upper surface of the most upper plate (3) of the bioreactor (1);

(c) adding fresh liquid culture medium via pipe (17) and second liquid inlet (16) and CO₂-containing gas via pipe (13) and first gas inlet (12) to the bioreactor (1);

(d) circulating liquid culture medium over the one or more plates (3) by collecting the liquid carbohydrate medium at second liquid outlet (8) located at the bottom (2c) of the bioreactor (1) and supplying it to the first liquid inlet (11) located at the top (2d) of the bioreactor via pipe (7), first pump (9) and pipe (10), to obtain an organic acid-containing

liquid medium and a gas depleted in CO₂;

(e) discharging the organic acid-containing liquid medium obtained in step (d) via the first liquid outlet (6) and pipe (5); and

(f) discharging the gas depleted in CO₂ obtained in step (d) via the first gas outlet (14) and pipe (15).

In a very preferred embodiment, the liquid culture medium entering via the first liquid inlet (11) located at the top (2d) of the bioreactor (1) is sprayed over the surface of the most upper plate (3) such that substantially all of the surface of the most upper plate (3) and the microorganism located thereon are wetted by the liquid culture medium.

The liquid culture medium serves, along with the gaseous CO₂, as nutrient medium for the anaerobic organic acid-producing microorganisms. These microorganisms convert the gaseous CO₂ and the nutrients in the liquid culture medium to organic acids.

When sufficient liquid culture medium is applied onto the most upper plate (3), liquid culture medium will start to drip down onto lower plates (3) and onto the microorganism located thereon, if more than one plate (3) is present in the bioreactor (1). Finally, the liquid culture medium reaches the bottom (2c) of the bioreactor (1) from which it is recycled to the first liquid inlet (11) located at the top (2d) of the bioreactor (1). This recycling process is performed in a continuous way. As will be understood by the person skilled in the art, during the recycling process of step (c), the composition of the liquid culture medium changes from a fresh liquid culture medium to an organic acid-containing liquid medium and the composition of the CO₂-containing gas becomes depleted in CO₂.

The inventor has found that the population of the anaerobic organic acid-producing microorganisms on the one or more plates (3) keeps growing during circulation step (d), thereby establishing a biofilm of microorganisms on the one or more plates (3). When the thickness of the biofilm exceeds a certain threshold value, part of the microorganism will be washed off the

at least one plate (3) by the liquid culture medium and will be recirculated over the at least one plate (3) along with the liquid culture medium.

This method can be applied in various ways. In a first embodiment, the process is operated batchwise, wherein in step (c) fresh liquid culture medium is added via pipe (17) and second liquid inlet (16) and CO₂-containing gas via pipe (13) and first gas inlet (12) to the bioreactor (1), after which second liquid inlet (16) and first gas inlet (12) are closed. Subsequently, the liquid culture medium is circulated over the at least one plate (3) in step (d).

As already described, during the recycling process of step (d), the composition of the liquid culture medium changes from a fresh liquid culture medium to an organic acid-containing liquid medium and the composition of the CO₂-containing gas becomes depleted in CO₂. After the required consumption of CO₂ in the bioreactor (1) has been reached, the organic acid-containing liquid medium is discharged via the first liquid outlet (6) and pipe (5) and the gas depleted in CO₂ is discharged via the first gas outlet (14) and pipe (15).

In a second embodiment, the process is operated, after a start-up phase, in a continuous way, wherein during the process as defined hereinbefore fresh liquid culture medium is continuously added via pipe (17) and second liquid inlet (16) to the bioreactor (1), wherein fresh CO₂-containing gas is continuously supplied via pipe (13) and first gas inlet (12) to the bioreactor (1), wherein organic acid-containing liquid medium is continuously discharged from the bioreactor (1) via the first liquid outlet (6) and pipe (5) and wherein the gas depleted in CO₂ is continuously discharged via the first gas outlet (14) and pipe (15) from the bioreactor (1). This continuous process requires that the liquid and gas streams that are continuously added to or removed from the bioreactor (1) are small as compared to the total gas and liquid volumes present inside the bioreactor (1). In this second embodiment, the first gas inlet (12) is located at the bottom (2c) of the bioreactor.

In a preferred embodiment, the CO₂-containing gas that is supplied to the bioreactor (1) in step (c) is selected from the group consisting of biogas, off-gas from a natural gas power plant, off-gas resulting from crude oil extraction, CO₂-containing gas from waste-water treatment, CO₂-containing gas from bio-ethanol production and combinations thereof.

In a very preferred embodiment, the CO₂-containing gas that is supplied to the bioreactor (1) in step (c) is biogas and the gas depleted in CO₂ is biogas enriched in CH 4. If the CO₂-containing gas that is supplied to the bioreactor (1) in step (c) is biogas, the gas enriched in CH₄ and depleted in CO₂ which is discharged from the bioreactor (1) in step (f) preferably contains at least 90 mol % CH₄, more preferably at least 95 mol % CH₄, even more preferably at least 98 mol % CH₄.

In a preferred embodiment, the CO₂-containing gas that is supplied to the bioreactor (1) in step (c) comprises 15 to 100 mol % CO₂, more preferably to 100 mol % CO₂, most preferably between 40 and 100 mol % CO₂.

Another preferred embodiment concerns a method for the anaerobic conversion of gaseous CO₂ and liquid culture medium to organic acids, said method comprising the steps of:

(a) providing a biogas production facility as defined hereinbefore;

(b) anaerobically digesting organic material in digester (20), resulting in CO₂-containing biogas;

(c) adding anaerobic organic acid-producing microorganisms to the upper surface of the plate (3) or to at least the upper surface of the most upper plate (3) of each bioreactor (1);

(d) adding fresh liquid culture medium via pipe (17) and second liquid inlet (16) to each bioreactor (1) and adding the CO₂-containing biogas of step (b) from the digester (20) via pipe (13) and first gas inlet (12) to each bioreactor (1);

(e) circulating liquid culture medium over the one or more plates (3) by collecting the liquid culture medium at liquid outlet (8) located at the bottom (2c) of each bioreactor (1) and supplying it to the first liquid inlet (11) located at the top (2d) of each bioreactor (1) via pipe (7), first pump (9) and pipe (10), to obtain an organic acid-containing liquid medium and a gas enriched in CH₄;

(f) discharging the organic acid-containing liquid medium obtained in step (e) via the first liquid outlet (6), pipe (5) and second pump (23) and liquid inlet (22) to the digester (20);

(g) discharging the gas enriched in CH₄ obtained in step (e) via the first gas outlet (14) and pipe (15).

This process can also be performed batchwise or in a continuous way.

Preferred examples of organic material encompass manure and biomass.

In this process, at least part of the CO₂-containing gas is biogas comprising CH₄ and CO₂ produced in the anaerobic digester (20). This CO₂-containing biogas is fed to the at least one bioreactor (1) where it is upgraded to biogas enriched in CH₄ and depleted in CO₂. The organic acid-containing liquid medium that is formed by the anaerobic organic acid-producing microorganisms by conversion of CO₂ and liquid culture medium is recycled to the digester (20). As already explained, this organic acid-containing liquid medium can also contain anaerobic organic acid-producing microorganisms washed off from the one or more plates (3).

The inventor has found that the organic acids produced in the bioreactor (1) can be advantageously used as nutrients by the anaerobic microorganisms that digest the organic material in the digester (20) to increase the yield of CH₄ per gram of organic material supplied to the digester (20).

As explained hereinbefore, dependent on the capacity of the digester (20) and the size of the bioreactor (1), the biogas production facility applied in the process can contain more than one bioreactor (1), such as 2 to 10 bioreactors (1).

In an embodiment, the CO₂-containing gas that is supplied to the bioreactor (1) via pipe (13) and first gas inlet (12) in step (d) is not only biogas produced in the anaerobic digester (20) but also comprises one or more CO₂-containing gases selected from the group consisting of offgas from a natural gas power plant, off-gas resulting from crude oil extraction, CO₂-containing gas from waste-water treatment and CO₂-containing gas from bio-ethanol production.

In a preferred embodiment, the CO₂-containing gases selected from the group consisting of off-gas from a natural gas power plant, off-gas resulting from crude oil extraction, CO₂-containing gas from waste-water treatment and CO₂-containing gas from bio-ethanol production comprises 15 to 100 mol % CO₂, more preferably 25 to 100 mol % CO₂, most preferably between 40 and 100 mol % CO₂.

Preferred organic acids that can be produced using the anaerobic organic acid-producing microorganisms include acetic acid, citric acid, succinic acid, fumaric acid, oxalic acid, and malic acid.

In a preferred embodiment, the anaerobic organic acid-producing microorganisms applied in the bioreactor (1), in the biogas production facility and in the methods as defined hereinbefore comprise organic acid-producing microorganisms selected from the group consisting of

Acetobacter, Gluconoacetobacter, Acidomonas, Gluconobacter, Sporomusa ovata (S. ovata), Clostridium ljungdahlii (C. ljungdahlii), Clostridium aceticum (C. aceticum), Moorella thermoacetica (M. thermoacetica), Acetobacterium woodii (A. woodii), Yarrowia lipolytica (Y. lipolytica), Candida lipolytica (C. lipolytica), Rhizopus oryzae (R. oryzae), Aspergillus niger(A. niger), Aspergillus terreus (A. terreus), Actinobacillus succinogenes (A. succinogenes), Anaerobiospirillum succiniciproducens (A. succiniciproducens), Mannheimia succiniciproducens (M. succiniciproducens), Corynebacterium glutamicum (C. glutamicum), recombinant Escherichia coli (E. coli) and combinations thereof.

In a very preferred embodiment, the anaerobic organic acid-producing microorganisms applied in the bioreactor (1), in the biogas production facility and in the methods as defined hereinbefore comprise succinic acid-producing microorganisms selected from the group consisting of Actinobacillus succinogenes (A. succinogenes), Anaerobiospirillum

succiniciproducens (A. succiniciproducens), Mannheimia succiniciproducens (M.

succiniciproducens), Corynebacterium glutamicum (C. glutamicum), recombinant Escherichia coli (E. coli) and combinations thereof. Even more preferably, the anaerobic organic acid-producing microorganisms comprise Actinobacillus succinogenes (A. succinogenes).

In a preferred embodiment, the gas enriched in CH₄ and depleted in CO₂ which is discharged from the at least one bioreactor (1) in step (g) contains at least 90 mol % CH 4, more preferably at least 95 mol % CH₄, even more preferably at least 98 mol % CH₄.

It is within the skills or the artisan to choose appropriate liquid culture media providing the required nutrients to the different anaerobic organic acid-producing microorganisms described hereinabove.

In a preferred embodiment, the (fresh) liquid culture medium comprises one or more of glucose, xylose, arabinose, galactose, maltose, fructose, sucrose, cellobiose, lactose, mannitol, arabitol, sorbitol, mannose, ribose, glycerol, pectin, beta-glucoside, gluconate, idonate, ascorbate, glucarate, galactarate, starch, corn steep liquor and 5-keto-glucanate.

In another preferred embodiment, the (fresh) liquid culture medium comprises a carbon source selected from the group consisting of glycerol and starch and combinations thereof, corn steep liquor as a nitrogen source, and optionally salts Salts that can advantageously be used in the liquid carbohydrate medium are NaCl and K₂HPO₄.

In a particularly preferred embodiment, the liquid culture medium comprises glycerol, corn steep liquor, NaCl and K₂HPO₄.

In another particularly preferred embodiment, the liquid culture medium comprises starch, corn steep liquor, NaCl and K₂HPO₄.

As will be understood by those skilled in the art, the remainder of the liquid culture medium consists of water.

Thus, the invention has been described by reference to certain embodiments discussed above. It will be recognized that these embodiments are susceptible to various modifications and alternative forms well known to those of skill in the art.

Furthermore, for a proper understanding of this document and its claims, it is to be understood that the verb ‘to comprise’ and its conjugations are used in its non-limiting sense to mean that items following the word are included, but items not specifically mentioned are not excluded. In addition, reference to an element by the indefinite article ‘a’ or ‘an’ does not exclude the possibility that more than one of the element is present, unless the context clearly requires that there be one and only one of the elements The indefinite article ‘a’ or ‘an’ thus usually means ‘at least one’.

### EXAMPLES

### Example 1: Conversion of CO₂ in a Bioreactor According to the Invention

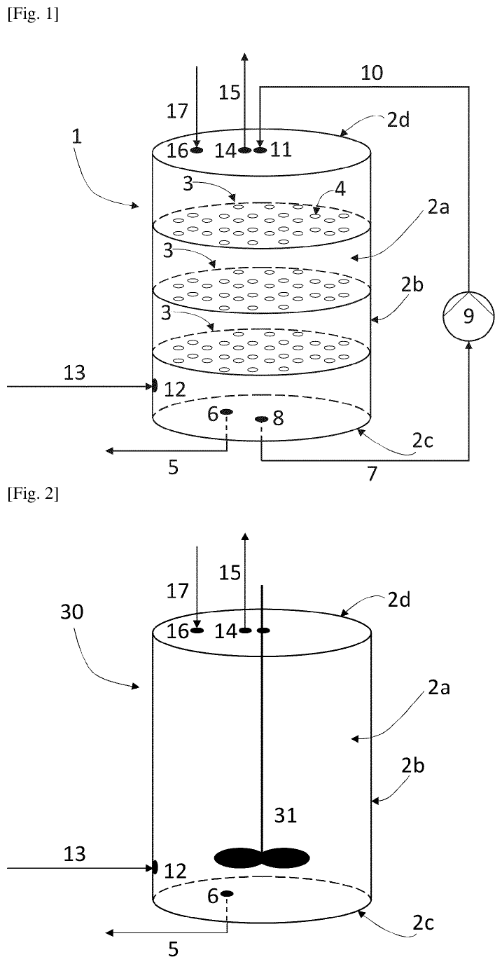

In a first experiment, the strain Actinobacillus succinogenus (DSM-22257) was obtained from DSMZ. This is an anaerobic succinic acid-producing microorganism, also producing other acids such as acetic acid. A bioreactor as depicted in FIG. 1 was provided. Bioreactor (1) had a size of 3 liter and comprised a cavity (2a), an outer wall (2b), a bottom (2c) and a top (2d).

The cavity comprised 9 plates (3) (ie more than the 3 plates indicated in FIG. 1) positioned perpendicularly to the outer wall at a distance of 2 cm from each other The plates (3) were made of plastic The plates (3) had more than 500 perforations (4) each with a size of about 1 to 2 mm The upper surface of the plates (3) was covered with anaerobic succinic acid-producing

microorganisms of the strain Actinobacillus succinogenus.

The bioreactor (1) further comprised a pipe (5) connected to a first liquid outlet (6) located at the bottom (2c) of the bioreactor for discharging liquid, a pipe (7) connected to a second liquid outlet (8) located at the bottom (2c) of the bioreactor (1) and to the inlet of a first pump (9), a pipe (10) connected to an outlet of the first pump (9) and to a first liquid inlet (11) located at the top (2d) of the bioreactor (1) for recycling liquid culture medium over the plates (3), a pipe (13) connected to a first gas inlet (12) located at the bottom (2c) of the bioreactor (1) for providing gaseous CO₂ to the bioreactor (1), a pipe (15) connected to a first gas outlet (14) for discharging gas from the bioreactor (1), and a pipe (17) connected to a second liquid inlet (16) for supplying fresh liquid culture medium to the bioreactor (1).

A total of 2500 ml of liquid medium was used of which, at any moment during the recycling process, about 500 ml was in the cavity (2a) and about 2000 ml in the recycling system [pipe (7), first pump (9) and pipe (10)].

The process was operated batchwise About 1000 ml of CO₂ gas was added to the bioreactor (1). The rate of circulation of the liquid medium over the plates (3) was 500 ml/hour. During the experiment, succinic acid and acetic acid were produced by Actinobacillus succinogenus NaOH (4M) was added to maintain a pH of 70 during the fermentation. The consumption of CO₂ in the reactor was measured at regular intervals.

The same anaerobic experiment was performed using a continuous stirred-tank reactor (CSTR) as a control, wherein the Actinobacillus succinogenus was distributed in the stirred liquid phase (see FIG. 2). In FIG. 2, the same numbering is used as in FIG. 1. Number (30) represents the CSTR and number (31) a stirrer. The CSTR had a volume of 3000 ml. Stirring took place at 150 rpm About 2500 ml of liquid medium was used. The process was operated batchwise. About 1000 ml of CO₂ gas was added at the bottom of the CSTR. Gas was recirculated (not shown in FIG. 2) over the liquid phase of the CSTR during fermentation by withdrawing it at the top (gas phase above the liquid phase) and by reintroducing it at the bottom of the CSTR Cultivation of Actinobacillus succinogenus in the liquid medium took place at a temperature of 37° C. During the experiment, succinic acid and acetic acid were produced by Actinobacillus succinogenus NaOH (4M) was added to maintain a pH of 70 during the fermentation. The consumption of CO₂ in the reactor was measured at regular intervals.

Tests were performed with pure CO₂ gas and with standard medium TSB as fresh liquid culture medium (see Table 1 for composition).

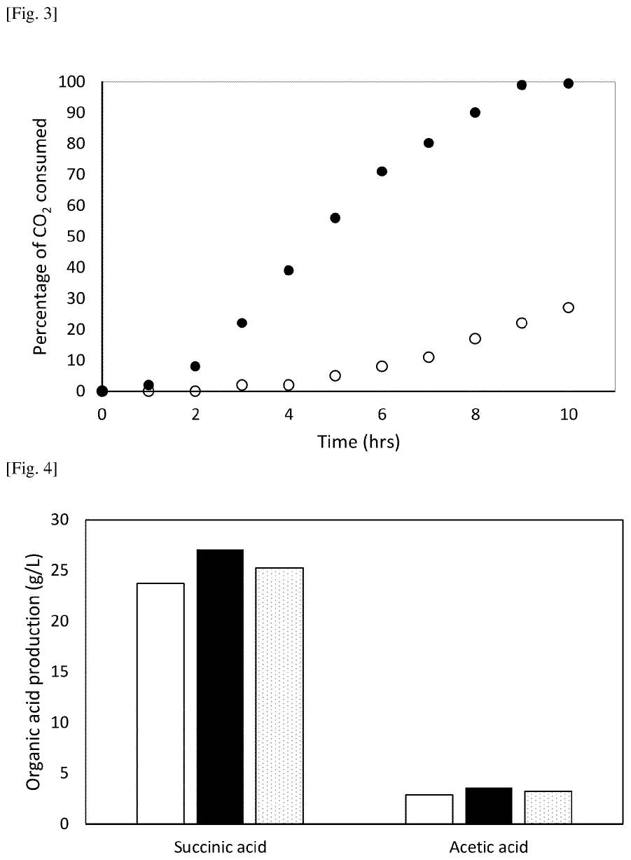

During the fermentation processes, the CO₂ consumption was monitored. The experiments were performed in duplicate Results are shown in FIG. 3, wherein solid (black) circles represent the consumption of CO₂ (as percentage of the initial amount of CO₂ present) as a function of time in the bioreactor (1) according to the invention and open (white) circles represent the consumption of CO₂ as a function of time in the CSTR.

Both process used the same amount of standard medium TSB and the same amount of CO₂ gas As can be seen in FIG. 3, the process in the bioreactor (1) according to the invention results in a much higher rate of CO₂ consumption than the process in the CSTR. After about 9 hours, all the CO₂ in the bioreactor (1) according to the invention had been consumed. After about 9 hours, the percentage of CO₂ consumed in the CSTR was only about 22%.

It was observed that during the anaerobic experiment in the bioreactor (1) according to the invention, a biofilm of Actinobacillus succinogenus had established on the plates of the bioreactor (1). Without wishing to be bound by theory, it is believed (i) that the higher rate of CO₂ consumption in the bioreactor (1) according to the invention as compared to the CSTR originates in the higher density of microorganisms that can be cultivated in the biofilm on the plates (3) as compared to the density of microorganism that can be obtained in submerged cultivation, and (ii) that the CO₂-containing gas can freely move through the perforations in the plates (3) of the bioreactor (1) and can freely contact the organic acid-producing microorganisms, hardly limited by the solubility of CO₂ in the liquid culture medium, whereas the limited solubility of CO₂ in the liquid culture medium is very relevant in submerged cultivation.

The above experiment in the bioreactor (1) according to the invention was repeated with two further liquid culture media SCB and GCB. See Table 1 for their compositions. Standard medium TSB is rather expensive Media SCB and GCB are, however, relatively inexpensive because the ingredients used are abundantly available Glycerol is for example a by-product in biodiesel production. Starch is available from potato waste Corn steep liquor broth is a by product of corn wet-milling.

TABLE 1
Composition of different liquid culture media (per liter water)

| Name | TSB^{(a)} | SCB^{(b)} | GCB^{(c)} |
|---|---|---|---|
| Peptone from casein | 17.0 | | |
| (g/liter) | | | |
| Peptone from | 3.0 | | |
| soymeal (g/liter) | | | |
| Glucose (g/liter) | 2.5 | | |
| Corn steep | | 10.0 | 10.0 |
| liquor(g/liter) | | | |
| Glycerol(g/liter) | | | 2.5 |
| Starch from | | 2.5 | |
| potato(g/liter) | | | |
| NaCl(g/liter) | 5 | 5.0 | 5.0 |
| K₂HPO₄ (g/liter) | 2.5 | 2.5 | 2.5 |
| Water (liter) | 1 | 1 | 1 |

^{(a)}TSB = Tryptone Soya Broth
^{(b)}SCB = Starch Corn steep liquor Broth
^{(c)}GCB = Glycerol Corn steep liquor Broth

The rate of CO₂ consumption in the bioreactor (1) according to the invention was similar for all three liquid media TSB, SCB and GCB. However, as can be seen in FIG. 4, liquid media SCB (black bars) and GCB (dotted bars) resulted in a slightly higher acid concentration (in gram succinic or acetic acid per liter of liquid medium) than TSB (white bars) after 24 hours.

The concentration of organic acid was measured by HPLC (Shimadzu, Kyoto, Japan) using an Aminex HPX-87H column (Bio-Rad, USA) and a refractive index detector (Shimadzu, Kyoto, Japan). The temperature of the column and detector was maintained at 65° C. The mobile phase was 0005 N H2SO4 at a flow rate of 0.55 ml/min.

### Example 2: Upgrading of Biogas in a Biogas Production Facility According to the Invention

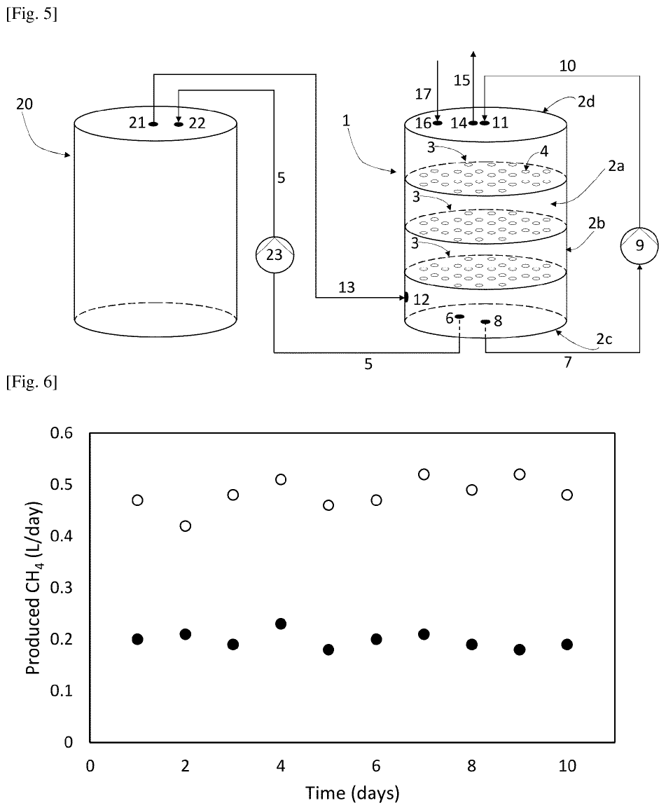

In a second experiment, the bioreactor (1) as described in Example 1 and an anaerobic digester (FIG. 5) were interconnected and operated at 37° C. in a constant-temperature environmental chamber.

The digester (20) was a CSTR with a total volume of 3000 ml About 2500 ml of liquid was used such that the CSTR had a headspace of about 500 ml The process was operated batchwise.

The process started with the addition of 2500 ml of water to the digester (20) followed by the addition of 25 g chicken manure. The chicken manure was obtained from Floradino Handels GmbH (Bergheim, Austria). The biogas produced was supplied to the bioreactor (1) as described in Example 1 using GCB as liquid culture medium, resulting in an organic acid-containing liquid medium and a biogas enriched in CH₄.

In the subsequent process, once a day, 100 ml of the liquid in the digester (20) was removed and 100 ml of the organic acid-containing liquid medium produced in the bioreactor (1) was added to the digester (20) together with 1 g of the chicken manure defined supra. The total liquid volume of 2500 ml in the digester (20) remained constant. The biogas produced in the digester (20) was supplied to the bioreactor (1) once a day The total amount of CH₄ produced in digester (20) was monitored.

A similar experiment (control) was performed using an identical digester (20) which was fed only with the chicken manure. In other words, organic acid-containing liquid medium produced in the bioreactor (1) was not supplied to the digester.

The reactors in both experiments were operated for 10 days and were continuously mixed. The produced biogas was sampled for quality analysis and collected in gas collection bottles for volume determination using liquid displacement. The biogas production rates were recorded daily Gas composition analysis was done using gas chromatography (Varian CP8410, GC) with a flame ionization detector.

Results are presented in FIG. 6, wherein solid (black) circles represent the daily production of CH₄ in the experimental setup without supplying organic acid-containing liquid medium produced in the bioreactor (1) and open (white) circles represent the daily production of CH₄ in the experimental setup with supplying organic acid-containing liquid medium produced in the bioreactor (1). It is clear from FIG. 6 that supplying organic acid-containing liquid medium results in improved CH₄ production and efficient CO₂ conversion.

1. Bioreactor (1) A bioreactor for the anaerobic conversion of gaseous CO₂ and a liquid culture medium to organic acids, said bioreactor comprising a cavity, an outer wall, a bottom and a top,
wherein the cavity comprises at least one plate having at least one perforation, wherein said at least one plate is positioned perpendicularly to the outer wall, wherein said bioreactor further comprising a pipe connected to a first liquid outlet located at the bottom of the bioreactor for discharging liquid, a pipe connected to a second liquid outlet located at the bottom of the bioreactor and to the inlet of a first pump, a pipe connected to an outlet of the first pump and to a first liquid inlet located at the top of the bioreactor for recycling liquid culture medium over the at least one plate, a pipe connected to a first gas inlet for providing CO₂-containing gas to the bioreactor, a pipe connected to a first gas outlet for discharging gas from the bioreactor and a pipe connected to a second liquid inlet for supplying fresh liquid culture medium to the bioreactor.

2. The bioreactor according to claim 1, comprising 2 to 500 plates having at least one perforation.

3. The bioreactor according to claim 1, wherein the at least one plate has multiple perforations and is a grid or a mesh screen.

4. The bioreactor according to claim 1, wherein the perforation or perforations have a size of between 0.5 and 100 mm, preferably between 1 and 2 mm.

5. The bioreactor according to claim 1, wherein said at least one plate comprises on its upper surface anaerobic organic acid-producing microorganisms.

6. A biogas production facility comprising a digester for the production of CO₂-containing biogas from organic material and at least one bioreactor according to claim 1, said digester comprising a gas outlet connected to pipe of the at least one bioreactor for supplying CO₂-containing biogas to the at least one bioreactor and a liquid inlet connected to pipe of the bioreactor for supplying organic acid-containing liquid medium to the digester via a second pump.

7. The biogas production facility according to claim 6, comprising 2 to 10 bioreactors.

8. The biogas production facility according to claim 6, wherein said at least one plate in each bioreactor comprises on its upper surface anaerobic organic acid-producing microorganisms.

9. A method for the anaerobic conversion of gaseous CO₂ and liquid culture medium to organic acids, said method comprising the steps of:
(a) providing a bioreactor according to claim 1;
(b) adding anaerobic organic acid-producing microorganisms to the upper surface of the plate or to at least the upper surface of the most upper plate of the bioreactor;
(c) adding fresh liquid culture medium via pipe and second liquid inlet and CO₂-containing gas via pipe and first gas inlet to the bioreactor;
(d) circulating liquid culture medium over the one or more plates by collecting the liquid carbohydrate medium at second liquid outlet located at the bottom of the bioreactor and supplying it to the first liquid inlet located at the top of the bioreactor via pipe, first pump and pipe, to obtain an organic acid-containing liquid medium and a gas depleted in CO₂;
(e) discharging the organic acid-containing liquid medium obtained in step (d) via the first liquid outlet and pipe; and
(f) discharging the gas depleted in CO₂ obtained in step (d) via the first gas outlet and pipe.

10. The method according to claim 9, wherein the CO₂-containing gas in step (c) is selected from the group consisting of biogas, off-gas from a natural gas power plant, off-gas resulting from crude oil extraction, CO₂-containing gas from waste-water treatment, CO₂-containing gas from bio-ethanol production and combinations thereof.

11. The method according to claim 9, wherein the CO₂-containing gas in step (c) is biogas, and wherein the gas depleted in CO₂ is biogas enriched in CH₄.

12. The method for the anaerobic conversion of gaseous CO₂ and liquid culture medium to organic acids, said method comprising the steps of:
(a) providing a biogas production facility according to claim 6;
(b) anaerobically digesting organic material in digester, resulting in CO₂-containing biogas;
(c) adding anaerobic organic acid-producing microorganisms to the upper surface of the plate or to at least the upper surface of the most upper plate of each bioreactor;
(d) adding fresh liquid culture medium via pipe and second liquid inlet to each bioreactor and adding the CO₂-containing biogas of step (b) from the digester via pipe and first gas inlet to each bioreactor;
(e) circulating liquid culture medium over the one or more plates by collecting the liquid culture medium at liquid outlet located at the bottom of each bioreactorand supplying it to the first liquid inlet located at the top of each bioreactor via pipe, pump and pipe, to obtain an organic acid-containing liquid medium and a gas enriched in CH₄;
(f) discharging the organic acid-containing liquid medium obtained in step (e) via the first liquid outlet, pipe and second pump and liquid inlet to the digester;
(g) discharging the gas enriched in CH₄ obtained in step (e) via the first gas outlet and pipe.

13. The method according to claim 12, wherein in step (d) also one or more CO₂-containing gases selected from the group consisting of off-gas from a natural gas power plant, off-gas resulting from crude oil extraction, CO₂-containing gas from waste-water treatment and CO₂-containing gas from bio-ethanol production are added via pipe and first gas inlet.

14. The bioreactor according to claim 5, wherein the anaerobic organic acid-producing microorganisms comprise organic acid-producing microorganisms selected from the group consisting of Acetobacter, Gluconoacetobacter, Acidomonas, Gluconobacter, Sporomusa ovata (S. ovata), Clostridium ljungdahlii (C. ljungdahlii), Clostridium aceticum (C. aceticum), Moorella thermoacetica (M. thermoacetica), Acetobacterium woodii (A. woodii), Yarrowia lipolytica (Y. lipolytica), Candida lipolytica (C. lipolytica), Rhizopus oryzae (R. oryzae), Aspergillus niger (A. niger), Aspergillus terreus (A. terreus), Actinobacillus succinogenes (A. succinogenes), Anaerobiospirillum succiniciproducens (A. succiniciproducens), Mannheimia succiniciproducens (M. succiniciproducens), Corynebacterium glutamicum (C. glutamicum), recombinant Escherichia coli (E. coli) and combinations thereof.

15. (canceled)

16. Method according to claim 9, wherein the fresh liquid culture medium comprises a carbon source selected from the group consisting of glycerol and starch and combinations thereof, corn steep liquor as a nitrogen source, and optionally salts.

17. Method according to claim 11, wherein the gas enriched in CH₄ contains at least 90 mol % CH₄, preferably at least 95 mol % CH₄, even more preferably at least 98 mol % CH₄.

18. The method according to claim 9, which is operated batchwise.

19. The method according to claim 9, which is operated in a continuous way.

20. The biogas production facility according to claim 8, wherein the anaerobic organic acid-producing microorganisms comprise organic acid-producing microorganisms selected from the group consisting of Acetobacter, Gluconoacetobacter, Acidomonas, Gluconobacter, Sporomusa ovata (S. ovata), Clostridium ljungdahlii (C. ljungdahlii), Clostridium aceticum (C. aceticum), Moorella thermoacetica (M. thermoacetica), Acetobacterium woodii (A. woodii), Yarrowia lipolytica (Y. lipolytica), Candida lipolytica (C. lipolytica), Rhizopus oryzae (R. oryzae), Aspergillus niger (A. niger), Aspergillus terreus (A. terreus), Actinobacillus succinogenes (A. succinogenes), Anaerobiospirillum succiniciproducens (A. succiniciproducens), Mannheimia succiniciproducens (M. succiniciproducens), Corynebacterium glutamicum (C. glutamicum), recombinant Escherichia coli (E. coli) and combinations thereof.

21. The method according to claim 9, wherein the anaerobic organic acid-producing microorganisms comprise organic acid-producing microorganisms selected from the group consisting of Acetobacter, Gluconoacetobacter, Acidomonas, Gluconobacter, Sporomusa ovata (S. ovata), Clostridium ljungdahlii (C. ljungdahlii), Clostridium aceticum (C. aceticum), Moorella thermoacetica (M. thermoacetica), Acetobacterium woodii (A. woodii), Yarrowia lipolytica (Y. lipolytica), Candida lipolytica (C. lipolytica), Rhizopus oryzae (R. oryzae), Aspergillus niger (A. niger), Aspergillus terreus (A. terreus), Actinobacillus succinogenes (A. succinogenes), Anaerobiospirillum succiniciproducens (A. succiniciproducens), Mannheimia succiniciproducens (M. succiniciproducens), Corynebacterium glutamicum (C. glutamicum), recombinant Escherichia coli (E. coli) and combinations thereof.
